(19) United States
(12) Patent Application Publication
(10) Pub. No.: US 2023/0010561 A1
(43) Pub. Date: Jan. 12, 2023

(54) SYSTEMS AND METHODS FOR PRESENTING CRUCIAL INFORMATION AND RECOMMENDATIONS

(71) Applicant: Alcon Inc., Fribourg (CH)

(72) Inventors: Judith Durá Díez, Berlin (DE); Horia Grecu, Berlin (DE); John Alfred Campin, Southlake, TX (US); George Hunter Pettit, Fort Worth, TX (US); Malgorzata Lukasik, Berlin (DE); Peter Zieger, Berlin (DE)

(21) Appl. No.: 17/810,698

(22) Filed: Jul. 5, 2022

Related U.S. Application Data
(60) Provisional application No. 63/219,853, filed on Jul. 9, 2021.

(51) Int. Cl. G16H 50/20; A61B 3/113; A61B 3/00

(52) U.S. Cl. CPC G16H 50/20; A61B 3/113; A61B 3/0041

(57) ABSTRACT

An ophthalmic system for analyzing data for a procedure includes a computer. The computer includes a memory, an interface device, and one or more processors that execute software. The memory stores procedure information for the procedure, and the interface device receives input and provide output. The one or more processors identify information from the procedure information that is crucial for the procedure, determine one or more recommendations to address the crucial information, and provide the crucial information and the one or more recommendations via the interface device.

### TECHNICAL FIELD

The present disclosure relates generally to ophthalmic systems, and more particularly to systems and methods for presenting crucial information and recommendations.

### BACKGROUND

Preparing for a procedure involves evaluating a large amount of data. Typically, the data is collected by a clinician instead of the surgeon who ultimately decides on the appropriate treatment. The surgeon often navigates through the data without guidance to find information crucial to the procedure. Crucial information may include information that is not easy to identify, e.g., outlier measurements and abnormal results. In some cases, the surgeon may fail to find crucial information to the attention of the surgeon.

This situation may lead to the surgeon being unaware of crucial information. When planning certain procedures, missing this information may lead to increased risk of complications and/or unsatisfactory results.

### BRIEF SUMMARY

In certain embodiments, an ophthalmic system for analyzing data for a procedure includes a computer. The computer includes a memory, an interface device, and one or more processors that execute software. The memory stores procedure information for the procedure, and the interface device receives input and provides output. The one or more processors identify information from the procedure information that is crucial for the procedure, determine one or more recommendations to address the crucial information, and provide the crucial information and the one or more recommendations via the interface device.

Embodiments may include none, one, some, or all of the following features: The one or more processors receive via the interface device a selection of a recommendation of the one or more recommendations, identify information from the procedure information that is crucial for the selected recommendation, determine one or more next recommendations to address the crucial information for the selected recommendation, and provide the crucial information for the selected recommendation and the one or more next recommendations via the interface device. The one or more processors receive via the interface device a change in the procedure, identify information from the procedure information that is crucial for the changed procedure, determine one or more next recommendations to address the crucial information for the changed procedure, and provide the crucial information for the changed procedure and the one or more next recommendations via the interface device. The one or more processors prioritize the one or more recommendations, and present the prioritized one or more recommendations. The crucial information includes aberrant information that comprises a value that departs from one or more acceptable values. The crucial information includes aberrant information that comprises a plurality of values of a parameter that differ beyond an acceptable variation. The crucial information comprises an eye measurement that is incompatible with the procedure. The one or more recommendations may comprise an IOL with which the eye measurement is compatible. The one or more recommendations may comprise an insertion procedure that makes an IOL compatible with the eye measurement. The one or more recommendations may comprise an IOL formula for calculating IOL parameters that is compatible with the eye measurement. The crucial information comprises a medical condition that is incompatible with the procedure. The one or more recommendations may comprise an IOL that is compatible with the medical condition. The one or more recommendations may comprise an IOL formula for calculating IOL parameters that is compatible with the medical condition. The procedure comprises a surgical procedure. The procedure comprises a diagnostic procedure.

In certain embodiments, a method for analyzing data for a procedure comprises: storing, by a memory, procedure information for the procedure; receiving, by an interface device, input and providing output; identifying, by one or more processors configured to execute software, information from the procedure information that is crucial for the procedure; determining, by the one or more processors, one or more recommendations to address the crucial information; and providing, by the one or more processors, the crucial information and the one or more recommendations via the interface device.

Embodiments may include none, one, some, or all of the following features: The method further comprises: receiving via the interface device a selection of a recommendation of the one or more recommendations; identifying information from the procedure information that is crucial for the selected recommendation; determining one or more next recommendations to address the crucial information for the selected recommendation; and providing the crucial information for the selected recommendation and the one or more next recommendations via the interface device. The method further comprises: receiving via the interface device a change in the procedure; identifying information from the procedure information that is crucial for the changed procedure; determining one or more next recommendations to address the crucial information for the changed procedure; and providing the crucial information for the changed procedure and the one or more next recommendations via the interface device. The method further comprises: prioritizing the one or more recommendations; and presenting the prioritized one or more recommendations.

In certain embodiments, an ophthalmic system for analyzing data for a procedure includes a computer. The computer includes a memory, an interface device, and one or more processors that execute software. The memory stores procedure information for the procedure that is a surgical procedure or a diagnostic procedure. The interface device receives input and provides output. The one or more processors identify information from the procedure information that is crucial for the procedure. The crucial information includes: aberrant information, the aberrant information comprising a value that departs from one or more acceptable values, and a plurality of values of a parameter that differ beyond an acceptable variation; an eye measurement that is incompatible with the procedure; and a medical condition that is incompatible with the procedure. The one or more processors determine one or more recommendations to address the crucial information. The recommendations includes: an IOL with which the eye measurement is compatible; an insertion procedure that makes an IOL compatible with the eye measurement; an IOL formula for calculating IOL parameters that is compatible with the eye measurement; an IOL that is compatible with the medical condition; and an IOL formula for calculating IOL parameters that is compatible with the medical condition. The one or more processors prioritize the one or more recommendations, and provide the crucial information and the one or more prioritized recommendations via the interface device. The one or more processors receive via the interface device a selection of a recommendation of the one or more recommendations, identify information from the procedure information that is crucial for the selected recommendation, determine one or more next recommendations to address the crucial information for the selected recommendation, and provide the crucial information for the selected recommendation and the one or more next recommendations via the interface device. The one or more processors receive via the interface device a change in the procedure, identify information from the procedure information that is crucial for the changed procedure, determine one or more next recommendations to address the crucial information for the changed procedure, and provide the crucial information for the changed procedure and the one or more next recommendations via the interface device.

### BRIEF DESCRIPTION OF THE DRAWINGS

FIG. 1 illustrates an example of an ophthalmic data analysis system that may provide crucial information and recommendations, according to certain embodiments;

FIG. 2 illustrates an example of a display that presents crucial information that includes an abnormal eye measurement;

FIG. 3 illustrates an example of a display that presents crucial information that includes inconsistent eye measurements from different devices;

FIG. 4 illustrates an example of a display that presents medical information that is incompatible with the procedure;

FIG. 5 illustrates an example of a display that presents an eye measurement that is incompatible with the procedure;

FIG. 6 illustrates an example of a display that presents medical information and an eye measurement that are incompatible with the procedure;

FIG. 7 illustrates an example of a display that presents a request for a type medical product; and

FIG. 8 illustrates an example of a method for presenting crucial information and recommendations that may be performed by the ophthalmic data analysis system of FIG. 1, according to certain embodiments.

### DESCRIPTION OF EXAMPLE EMBODIMENTS

Referring now to the description and drawings, example embodiments of the disclosed apparatuses, systems, and methods are shown in detail. The description and drawings are not intended to be exhaustive or otherwise limit the claims to the specific embodiments shown in the drawings and disclosed in the description. Although the drawings represent possible embodiments, the drawings are not necessarily to scale and certain features may be simplified, exaggerated, removed, or partially sectioned to better illustrate the embodiments.

In certain embodiments, a data analysis system identifies information that is crucial for a procedure, e.g., a surgical procedure, and determines one or more recommendations to address the crucial information. The system outputs the crucial information and recommendations to a user (e.g., clinician, technician, or surgeon) via an interface device. The system thus notifies the user of crucial information and recommendations, which may decrease the risk of complications and/or unsatisfactory results. The data analysis may be performed for any suitable purpose. For example, a user may use the crucial information and recommendations to plan an ophthalmic surgery, to detect an abnormality during a diagnostic procedure, or to identify a faulty measurement.

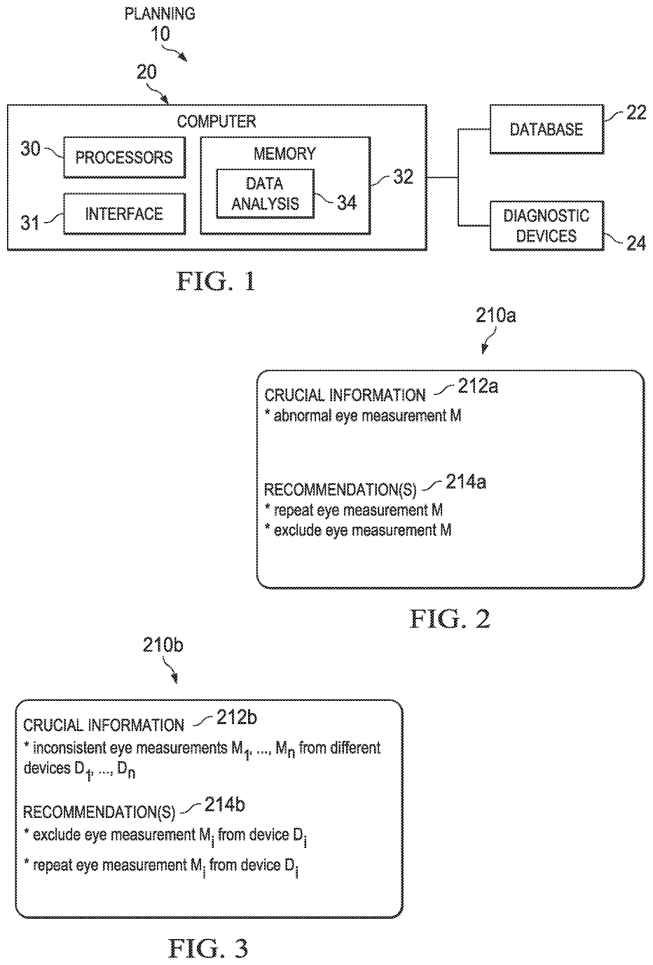

FIG. 1 illustrates an example of an ophthalmic data analysis system 10 that may provide crucial information and recommendations, according to certain embodiments. In the illustrated example, data analysis system 10 includes a computer 20, in communication with a database 22 and one or more diagnostic devices 24. Computer 20 includes one or more processors 30, an interface 31 (such as a computer display), and a memory 32, which stores applications such as data analysis software 34.

In certain embodiments, computer 20 receives via an interface device a selection of a procedure, e.g., a surgical procedure (e.g., inserting an intraocular lens (IOL)). Computer 20 accesses procedure information, e.g., surgical planning information for the surgical procedure (e.g., axial length AL of eye), from memory 32. The information may be received from database 22 and/or one or more diagnostic devices 24. Computer 20 executes data analysis software 34 to identify information that is crucial for the procedure (e.g., axial length too long), and to determine one or more recommendations to address the crucial information (e.g., recommend a specific IOL calculation formula). Computer 20 then outputs the crucial information and the recommendations via interface 31.

In certain examples, the procedure information may include any suitable information related to the procedure, e.g., an ophthalmic surgical procedure. For example, the information may include values for specific parameters, such as measurements of: biometric dimensions of an eye (e.g., axial length); physiologic data (e.g., intraocular pressure); refractive characteristics of an eye (e.g., refractive indexes, eye aberrations, tomography); and/or anatomic characteristic of the eye (e.g., pupil size under different light conditions, limbus size). As another example, the information may include medical information, such as ophthalmic diagnostic information (e.g., keratoconus, glaucoma); patient demographics (e.g., female); clinical information (e.g., lab test results); symptoms (e.g., watery eyes); current treatments (e.g., contact lenses); medications (e.g., high blood pressure medication); medical history (e.g., retinal detachment); previous ocular surgery (e.g., myopic corneal refractive surgery such as LASIK surgery); and/or medical images (e.g. retinal image, 3D lens image). As yet another example, the information may include patient and/or surgeon preferences, such as preferences for a planning and/or calculation tool (e.g., application of a particular IOL calculation formula for specific eye characteristics), surgical technique, a medical product (e.g., requested advanced-technology intraocular lens (AT-IOL)), or other preference.

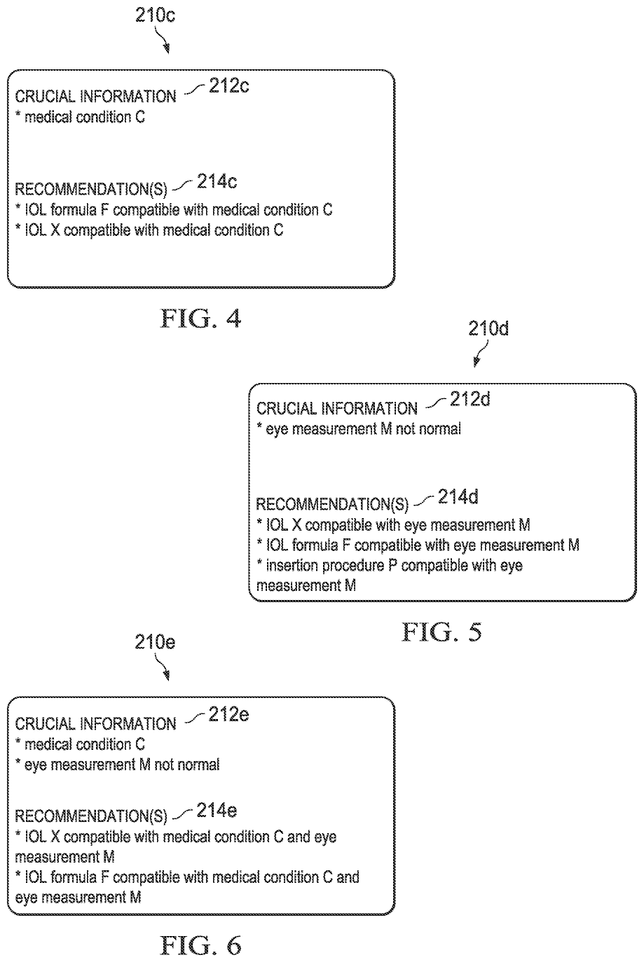

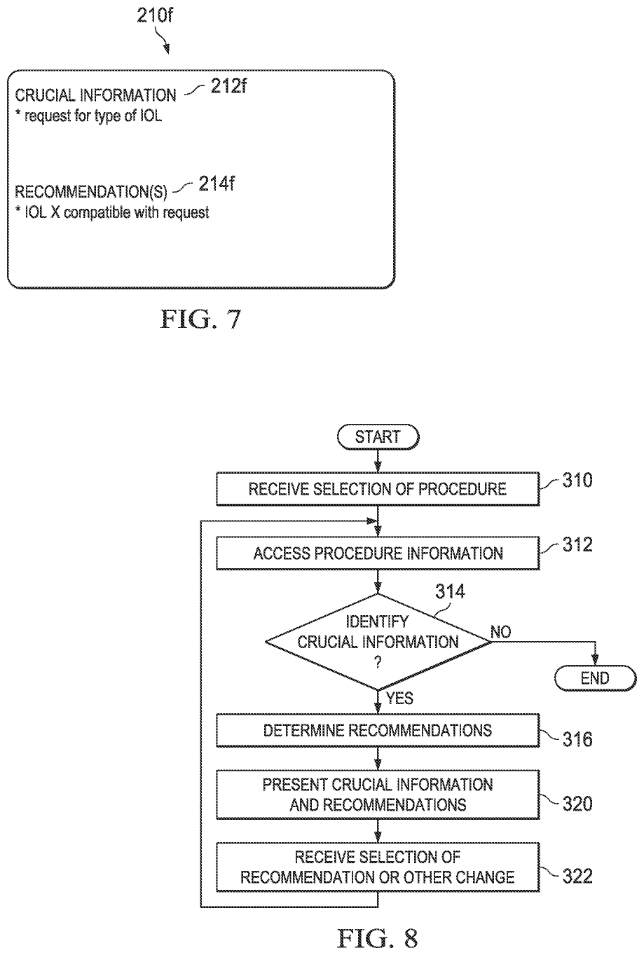

Crucial information may comprise information that potentially negatively affects the outcome of the procedure. For example, crucial information for a surgical procedure may include factors that may yield surgical complications or unsatisfactory results. Examples of crucial information include: aberrant information (examples described with respect to FIGS. 2 and 3), a factor that is incompatible with the procedure (examples described with respect to FIGS. 4 to 6), a factor that is incompatible with medical product (examples described with respect to FIG. 7), or other information that potentially negatively affects the outcome of the procedure.

Computer 20 may identify crucial information and determine the recommendations for responding to the crucial information in any suitable manner. In certain embodiments, computer 20 stores search criteria used to identify crucial information, such as unacceptable or incompatible values, as described in more detail below. Computer 20 also stores recommendations for responding to situations described by the identified crucial information, as described in more detail below. In the embodiments, computer 20 searches the procedure information using the search criteria to identify crucial information, and then accesses the stored recommendations to determine the recommendations for the identified crucial information.

Computer 20 outputs the results of the data analysis via interface 31. In certain embodiments, the data analysis is used to plan an ophthalmic procedure (e.g., a surgical or diagnostic procedure). In some cases, the data analysis is used to perform a surgical procedure. The crucial information and recommendations may be used to make selections for the procedure (e.g., selections of a calculation recommendation, surgical technique, and/or medical product), which may be included in the results. The results may be output to the user via interface 31 and/or sent to a surgical system via interface 31. In other cases, the data analysis is used to perform a diagnostic procedure. The crucial information may be used to detect an abnormality of the patient or to identify a faulty measurement, and the recommendations may suggest a diagnosis for the abnormality or may identify a potentially defective measuring device. The results may be output to the user via an interface 31 that allows the user to readily address the situation.

Turning to the components in communication with computer 20, database 22 may be any suitable memory that can store procedure information. A diagnostic device 24 may be any suitable device that measures one or more features of an eye. Examples of diagnostic devices include topographers, wavefront aberrometers, optical coherence tomography (OCT) devices with applicability for 2D and 3D, biometry or retina imaging devices, Scheimpflug imaging devices, or other devices that measure a feature of an eye.

FIGS. 2 and 3 illustrate examples of displays 210 (210a, 210b) that present aberrant information. Aberrant information may comprise a value of a parameter that departs from acceptable values for the parameter. For example, a value of a parameter may depart from one or more predefined acceptable values, or a plurality of values of the same parameter may differ beyond an acceptable variation.

Computer 20 may identify aberrant information in any suitable manner. In certain embodiments, computer 20 may access search criteria describing one or more acceptable values (e.g., given as one or more values or a range of values) for a parameter, scan the procedure information for instances of the parameter, and detect a value that is not acceptable. In certain embodiments, computer 20 may access search criteria describing an acceptable variation for the values of a measurement, scan the procedure information for values of the measurement, and detect values that vary beyond the acceptable variation. Computer 20 may also access recommendations to provide in response to finding the aberrant information.

FIG. 2 illustrates an example of a display 210a that presents crucial information 212a that includes an abnormal eye measurement M. Examples of abnormal eye measurements include: (1) An axial length AL that is outside of an acceptable range, e.g., AL=45.35 millimeters (mm), where the acceptable range is 12 to 40 mm. (2) An axial length with a standard deviation SD that is outside of an acceptable range, e.g., AL=28.50±0.05 mm, where the threshold standard deviation SD is 0.03 mm.

Recommendations 214a include repeat eye measurement M and exclude eye measurement M. Repeating eye measurement M may be prioritized over excluding eye measurement M because having the eye measurement M may be preferable to not having the measurement M. In other cases, excluding eye measurement M may be prioritized over repeating eye measurement M because the eye measurement M might not be important for the procedure.

FIG. 3 illustrates an example of a display 210b that presents crucial information 212b that includes inconsistent eye measurements M₁, . . . , Mₙ from different devices D₁, . . . , Dₙ. An example of inconsistent eye measurements includes measurements that exceed the acceptable distribution of measurements. For example, given axial length AL measurements of 28.50 mm, 28.53 mm, and 28.60 mm, with an acceptable distribution of ±0.03 mm, measurement 28.60 mm exceeds the acceptable distribution.

Recommendations 214b include exclude eye measurement Mᵢ from device Dᵢ. and repeat eye measurement Mᵢ from device Dᵢ. Excluding eye measurement M may be prioritized over repeating eye measurement M because the additional eye measurement M might not be necessary.

FIGS. 4 to 6 illustrate examples of displays 210 (210c to 210e) that present crucial information that is incompatible with the procedure, e.g., information that indicates a potential negative outcome of the procedure. Computer 20 may identify incompatible crucial information in any suitable manner. In certain embodiments, computer 20 may access search criteria describing conditions and/or measurements incompatible with the procedure, scan the procedure information for instances of the conditions and/or measurements, and detect an incompatible condition and/or measurement. Computer 20 may also access recommendations to provide in response to finding the incompatible condition and/or measurement.

FIG. 4 illustrates an example of a display 210c that presents medical information that is incompatible with the procedure, e.g., information that indicates a patient might not be a good candidate for a procedure or a medical product. In the example, critical information 212c includes a medical condition C. Examples of medical condition C include keratoconus or a prior corneal refractive surgical procedure. Recommendations 214c include an IOL formula F and an IOL X compatible with medical condition C. Examples of IOL formula F include Holladay II for keratoconus or Barret True K for the prior corneal refractive surgical procedure.

FIG. 5 illustrates an example of a display 210d that presents an eye measurement that is incompatible with the procedure. Critical information 212d includes eye measurement M that is within the normal range, but outside of standard values. Recommendations 214d include an IOL, an IOL formula F, and an insertion procedure P compatible with eye measurement M. For example, if an axial length AL measurement of 29.50 mm exceeds a long eye threshold of 29.00 mm, an IOL formula F for long eyes may be recommended.

FIG. 6 illustrates an example of a display 210e that presents medical information and an eye measurement that are incompatible with the procedure. Critical information 212e includes medical condition C and eye measurement M not normal. Recommendations 214e include an IOL and an IOL formula F compatible with medical condition C and eye measurement M.

FIG. 7 illustrates an example of a display 210f that presents a request for a type medical product, e.g., an IOL such as a multifocal IOL. In the example, critical information 212f includes the request. Recommendations 214f include an IOL X compatible with the request.

Other examples include: (1) If the request is for a multifocal IOL, the recommendation customizes the results based on the request, e.g., show Angle Kappa (Kc) of Kc=14.50° for surgeon evaluation. (2) If the request is for an IOL for an eye with a longer axial length, the recommendation suggests an IOL that supports powers for longer axial lengths. (3) If the request is for a multifocal IOL and the pupil of the patient's eye is not sufficiently centered, the recommendation suggests a more compatible IOL, such as a monofocal IOL. (4) If the request is for a high refraction index IOL and the patient information indicates possibility of negative dysphotopsias (e.g., previous surgery for corneal incisions), the recommendation suggests a different IOL and/or different placement of IOL (e.g., in front of capsule). (5) If the request is for a standard IOL, the recommendation suggests a premium IOL that is appropriate for the patient's eye.

FIG. 8 illustrates an example of a method for presenting crucial information and recommendations that may be performed by ophthalmic data analysis system 10 of FIG. 1, according to certain embodiments. The method starts at step 310, where computer 20 receives the selection of a procedure, e.g., a surgical procedure, via an interface device. For example, the procedure may be inserting an intraocular lens (IOL) into an eye. Computer 20 accesses procedure information for the procedure from a memory at step 312. Procedure information may include, e.g., medical information and/or measurements of the eye.

Computer 20 may identify crucial information at step 314. Crucial information may include, e.g., medical information that is incompatible with the procedure and/or a measurement that has an unacceptable value. If computer 20 identifies crucial information, the method proceeds to step 316. If computer 20 does not identify crucial information, the method ends.

Computer 20 determines recommendations at step 316. For example, if a medical condition is crucial to IOL implantation, computer 20 may recommend: an IOL formula for calculating IOL parameters that is compatible with the medical condition; and/or an IOL that is compatible with the medical condition. In certain embodiments, computer 20 may prioritize the recommendations and present the prioritized list of recommendations.

Computer 20 presents the crucial information and recommendations at step 320. Computer 20 may receive a selection of a recommendation or other change in the procedure at step 322. A change in the procedure may be, e.g., a change to a different IOL that was not recommended by the system but that the surgeon selected. The method returns to step 312 to access the procedure information.

If computer 20 identifies information that is crucial for the selected recommendation or other change at step 314, computer 20 determines one or more next recommendations to address the crucial information at step 316. For example, if an eye measurement is incompatible with a selected IOL, computer 20 may recommend: an IOL with which the eye measurement is compatible; and/or an incision procedure that makes the selected IOL compatible with the eye measurement. Computer 20 presents the crucial information and the one or more next recommendations via an interface device at step 320. If computer 20 does not identify crucial information at step 314, the method ends.

A component (such as computers 20 and 103) of the systems and apparatuses disclosed herein may include an interface, logic, and/or memory, any of which may include computer hardware and/or software. An interface can receive input to the component and/or send output from the component, and is typically used to exchange information between, e.g., software, hardware, a peripheral device, a user, and/or other external entity. A user may use an interface, such as a Graphical User Interface (GUI) or an interface device, to interact with (e.g., provide input and/or receive output) a computer. Examples of an interface device include input and/or output devices, such as a keyboard, mouse, touchpad, touchscreen, microphone, computer display, foot pedal, gesture sensor, and/or speaker.

Logic can perform operations of the component. Logic may include one or more electronic devices that process data, e.g., execute instructions to generate output from input. Examples of such an electronic device include a computer, processor, microprocessor (e.g., a Central Processing Unit (CPU)), and computer chip. Logic may include computer software that encodes instructions capable of being executed by the electronic device to perform operations. Examples of computer software include a computer program, application, and operating system.

A memory can store information and may comprise tangible, computer-readable, and/or computer-executable storage medium. Examples of memory include computer memory (e.g., Random Access Memory (RAM) or Read Only Memory (ROM)), mass storage media (e.g., a hard disk), removable storage media (e.g., a Compact Disk (CD) or Digital Video or Versatile Disk (DVD)), database, network storage (e.g., a server), and/or other computer-readable media. Particular embodiments may be directed to memory encoded with computer software.

Although this disclosure has been described in terms of certain embodiments, modifications (such as changes, substitutions, additions, omissions, and/or other modifications) of the embodiments will be apparent to those skilled in the art. Accordingly, modifications may be made to the embodiments without departing from the scope of the invention. For example, modifications may be made to the systems and apparatuses disclosed herein. The components of the systems and apparatuses may be integrated or separated, or the operations of the systems and apparatuses may be performed by more, fewer, or other components, as apparent to those skilled in the art. As another example, modifications may be made to the methods disclosed herein. The methods may include more, fewer, or other steps, and the steps may be performed in any suitable order, as apparent to those skilled in the art.

To aid the Patent Office and readers in interpreting the claims, Applicants note that they do not intend any of the claims or claim elements to invoke 35 U.S.C. § 112(f), unless the words “means for” or “step for” are explicitly used in the particular claim. Use of any other term (e.g., “mechanism,” “module,” “device,” “unit,” “component,” “element,” “member,” “apparatus,” “machine,” “system,” “processor,” or “controller”) within a claim is understood by the applicants to refer to structures known to those skilled in the relevant art and is not intended to invoke 35 U.S.C. § 112(f).

What is claimed:

1. An ophthalmic system for analyzing data for a procedure, comprising a computer comprising:
a memory configured to store procedure information for the procedure;
an interface device configured to receive input and provide output; and
one or more processors configured to execute software to:
identify information from the procedure information that is crucial for the procedure;
determine one or more recommendations to address the crucial information; and
provide the crucial information and the one or more recommendations via the interface device.

2. The ophthalmic system of claim 1, the one or more processors further configured to:
receive via the interface device a selection of a recommendation of the one or more recommendations;
identify information from the procedure information that is crucial for the selected recommendation;
determine one or more next recommendations to address the crucial information for the selected recommendation; and
provide the crucial information for the selected recommendation and the one or more next recommendations via the interface device.

3. The ophthalmic system of claim 1, the one or more processors further configured to:
receive via the interface device a change in the procedure;
identify information from the procedure information that is crucial for the changed procedure;
determine one or more next recommendations to address the crucial information for the changed procedure; and
provide the crucial information for the changed procedure and the one or more next recommendations via the interface device.

4. The ophthalmic system of claim 1, the one or more processors further configured to:
prioritize the one or more recommendations; and
present the prioritized one or more recommendations.

5. The ophthalmic system of claim 1, the crucial information comprising aberrant information, the aberrant information comprising a value that departs from one or more acceptable values.

6. The ophthalmic system of claim 1, the crucial information comprising aberrant information, the aberrant information comprising a plurality of values of a parameter that differ beyond an acceptable variation.

7. The ophthalmic system of claim 1, the crucial information comprising an eye measurement that is incompatible with the procedure.

8. The ophthalmic system of claim 7, the one or more recommendations comprising:
an IOL with which the eye measurement is compatible.

9. The ophthalmic system of claim 7, the one or more recommendations comprising:
an insertion procedure that makes an IOL compatible with the eye measurement.

10. The ophthalmic system of claim 7, the one or more recommendations comprising:
an IOL formula for calculating IOL parameters that is compatible with the eye measurement.

11. The ophthalmic system of claim 1, the crucial information comprising a medical condition that is incompatible with the procedure.

12. The ophthalmic system of claim 11, the one or more recommendations comprising:
an IOL that is compatible with the medical condition.

13. The ophthalmic system of claim 11, the one or more recommendations comprising:
an IOL formula for calculating IOL parameters that is compatible with the medical condition.

14. The ophthalmic system of claim 1, the procedure comprising a surgical procedure.

15. The ophthalmic system of claim 1, the procedure comprising a diagnostic procedure.

16. A method for analyzing data for a procedure, comprising:
storing, by a memory, procedure information for the procedure;
receiving, by an interface device, input and providing output;
identifying, by one or more processors configured to execute software, information from the procedure information that is crucial for the procedure;
determining, by the one or more processors, one or more recommendations to address the crucial information; and
providing, by the one or more processors, the crucial information and the one or more recommendations via the interface device.

17. The method of claim 16, further comprising:
receiving via the interface device a selection of a recommendation of the one or more recommendations;
identifying information from the procedure information that is crucial for the selected recommendation;
determining one or more next recommendations to address the crucial information for the selected recommendation; and
providing the crucial information for the selected recommendation and the one or more next recommendations via the interface device.

18. The method of claim 16, further comprising:
receiving via the interface device a change in the procedure;
identifying information from the procedure information that is crucial for the changed procedure;
determining one or more next recommendations to address the crucial information for the changed procedure; and
providing the crucial information for the changed procedure and the one or more next recommendations via the interface device.

19. The method of claim 16, further comprising:
prioritizing the one or more recommendations; and
presenting the prioritized one or more recommendations.

20. An ophthalmic system for analyzing data for a procedure, comprising a computer comprising:
a memory configured to store procedure information for the procedure, the procedure comprising a surgical procedure or a diagnostic procedure;
an interface device configured to receive input and provide output; and
one or more processors configured to execute software to:
identify information from the procedure information that is crucial for the procedure, the crucial information comprising: aberrant information, the aberrant information comprising a value that departs from one or more acceptable values, and a plurality of values of a parameter that differ beyond an acceptable variation; an eye measurement that is incompatible with the procedure; and a medical condition that is incompatible with the procedure;
determine one or more recommendations to address the crucial information, the one or more recommendations comprising: an IOL with which the eye measurement is compatible; an insertion procedure that makes an IOL compatible with the eye measurement; an IOL formula for calculating IOL parameters that is compatible with the eye measurement; an IOL that is compatible with the medical condition; and an IOL formula for calculating IOL parameters that is compatible with the medical condition;
prioritize the one or more recommendations;
provide the crucial information and the one or more prioritized recommendations via the interface device;
receive via the interface device a selection of a recommendation of the one or more recommendations;
identify information from the procedure information that is crucial for the selected recommendation;
determine one or more next recommendations to address the crucial information for the selected recommendation;
provide the crucial information for the selected recommendation and the one or more next recommendations via the interface device;
receive via the interface device a change in the procedure;
identify information from the procedure information that is crucial for the changed procedure;
determine one or more next recommendations to address the crucial information for the changed procedure; and
provide the crucial information for the changed procedure and the one or more next recommendations via the interface device.
